(19) United States
(12) Patent Application Publication
(10) Pub. No.: US 2025/0160858 A1
(43) Pub. Date: May 22, 2025

(54) HIGH PRESSURE PUMP FOR THROMBECTOMY SYSTEM

(71) Applicant: Boston Scientific Scimed, Inc., Maple Grove, MN (US)

(72) Inventors: Keval Shah, Maple Grove, MN (US); Mark Alfred Hilse, Somerset, WI (US); Thomas A. Wrobel, Alexandria, MN (US); Brian Beaubien, St. Paul, MN (US); Jacob Stoesz, Golden Valley, MN (US); David Skodje, Minneapolis, MN (US)

(73) Assignee: Boston Scientific Scimed, Inc., Maple Grove, MN (US)

(21) Appl. No.: 18/954,062

(22) Filed: Nov. 20, 2024

Related U.S. Application Data
(60) Provisional application No. 63/601,445, filed on Nov. 21, 2023.

(51) Int. Cl. A61B 17/22; A61B 17/00

(52) U.S. Cl. CPC A61B 17/22; A61B 2017/00477; A61B 2017/00539; A61B 2017/00836; A61B 2017/00938; A61B 2017/00946

(57) ABSTRACT

An example medical device is disclosed. An example high pressure pump for use in thrombectomy system includes a cylinder having an upper region, a lower region, and a fluid pumping chamber that includes a piston rod extending within at least a portion of the fluid pumping chamber, wherein the piston rod is configured to pump fluid through a high pressure fluid supply tube. The high pressure pump also includes a high pressure connector having a first end region and a second end region, wherein the second end is coupled to the lower region of the cylinder and a high pressure seal assembly is positioned between the second end of the high pressure connector and the cylinder, and wherein the high pressure seal assembly includes a T-plate and a crush washer.

### CROSS-REFERENCE TO RELATED APPLICATIONS

This application claims the benefit of priority under 35 U.S.C. § 119 of U.S. Provisional Application No. 63/601,445, filed Nov. 21, 2023, the entire disclosure of which is hereby incorporated by reference.

### TECHNICAL FIELD

The disclosure is directed to thrombectomy systems. More particularly, the disclosure is directed to a thrombectomy system with a high pressure pump.

### BACKGROUND

Thrombectomy is a procedure for removing thrombus from the vasculature of a patient. Mechanical and fluid-based systems can be used to remove thrombus. With fluid-based systems, an infusion fluid may be infused to a treatment area of a vessel with a catheter to dislodge the thrombus. In some instances, an effluent (e.g., the infusion fluid and/or blood) including the dislodged thrombus may be extracted from the vessel through the catheter. Of the known thrombectomy systems and methods, there is an ongoing need to provide alternative configurations of thrombectomy catheters and systems, as well as methods of operating such thrombectomy systems.

### SUMMARY

This disclosure provides design, material, manufacturing method, and use alternatives for medical devices. An example high pressure pump for use in thrombectomy system includes a cylinder having an upper region, a lower region, and a fluid pumping chamber that includes a piston rod extending within at least a portion of the fluid pumping chamber, wherein the piston rod is configured to pump fluid through a high pressure fluid supply tube. The high pressure pump also includes a high pressure connector having a first end region and a second end region, wherein the second end is coupled to the lower region of the cylinder and a high pressure seal assembly is positioned between the second end of the high pressure connector and the cylinder, and wherein the high pressure seal assembly includes a T-plate and a crush washer.

Alternatively or additionally to any of the examples above, wherein the T-plate is positioned radially outward of the crush washer.

Alternatively or additionally to any of the examples above, wherein the T-plate includes a first radially outward facing surface and a second radially inward facing surface, and wherein the crush washer is positioned between the second radially inward facing surface and the cylinder.

Alternatively or additionally to any of the examples above, wherein the first radially outward facing surface of the T-plate is configured to engage the high pressure connector.

Alternatively or additionally to any of the examples above, wherein the second end region of the high pressure connector includes a first threaded region, and wherein the first threaded region of the high pressure connector is configured to engage a second threaded region of the cylinder, and wherein rotation of the first threaded region of the high pressure connector along the second threaded region of the cylinder engages the second radially inward facing surface of the T-plate with the crush washer.

Alternatively or additionally to any of the examples above, wherein the engagement of the second radially inward facing surface of the T-plate with the crush washer deforms the crush washer from a first configuration to a second configuration different from the first configuration.

Alternatively or additionally to any of the examples above, wherein the crush washer includes a first central aperture, and wherein the high pressure fluid supply tube extends through the first central aperture of the crush washer, and wherein the deformation of the crush washer from the first configuration to the second configuration seals the crush washer onto the high pressure fluid supply tube.

Alternatively or additionally to any of the examples above, wherein a proximal end region of the high pressure fluid supply includes a filter and wherein at least a portion of the filter is positioned within the fluid pumping chamber.

Alternatively or additionally to any of the examples above, wherein the cylinder further includes a check ball seat configured to receive a check ball positioned within the fluid pumping chamber, and wherein the filter is positioned between the check ball and the piston rod.

Alternatively or additionally to any of the examples above, wherein the crush washer includes a first cross-sectional thickness and a second cross-sectional thickness different from the first cross-sectional thickness.

Alternatively or additionally to any of the examples above, wherein the T-plate includes a stem having a lumen, and wherein the high pressure fluid supply tube is configured to extend within the lumen of the stem.

Alternatively or additionally to any of the examples above, wherein at least a portion of the stem is configured to extend into a lumen of the high pressure connector.

Alternatively or additionally to any of the examples above, further comprising a liner extending along at least a portion of an inner surface of the cylinder.

Alternatively or additionally to any of the examples above, wherein the liner is formed from ultra-high-molecular-weight polyethylene.

Another example thrombectomy system includes a fluid inflow pump coupled to a thrombectomy catheter and driven by a console, wherein the fluid inflow pump includes a cylinder having an upper region, a lower region, and a fluid pumping chamber. The fluid inflow pump also includes a piston rod extending within at least a portion of the fluid pumping chamber, wherein the piston rod is configured to pump fluid through a high pressure fluid supply tube. The fluid inflow pump also includes a high pressure connector having a first end region and a second end region, wherein the second end is coupled to the lower region of the cylinder and a high pressure seal assembly is positioned between the second end of the high pressure connector and the cylinder, and wherein the high pressure seal assembly includes a T-plate and a crush washer.

Alternatively or additionally to any of the examples above, wherein the T-plate includes a first radially outward facing surface and a second radially inward facing surface, and wherein the crush washer is positioned between the second radially inward facing surface and the cylinder.

Alternatively or additionally to any of the examples above, wherein the second end region of the high pressure connector includes a first threaded region, and wherein the first threaded region of the high pressure connector is configured to engage a second threaded region of the cylinder, and wherein rotation of the first threaded region of the high pressure connector along the second threaded region of the cylinder engages the second radially inward facing surface of the T-plate with the crush washer.

Alternatively or additionally to any of the examples above, wherein the engagement of the second radially inward facing surface of the T-plate with the crush washer deforms the crush washer from a first configuration to a second configuration different from the first configuration.

Alternatively or additionally to any of the examples above, wherein the crush washer includes a first central aperture, and wherein the high pressure fluid supply tube extends through the first central aperture of the crush washer, and wherein the deformation of the crush washer from the first configuration to the second configuration seals the crush washer onto the high pressure fluid supply tube.

An example thrombectomy system includes a fluid inflow pump coupled to a thrombectomy catheter and driven by a console, wherein the fluid inflow pump includes a cylinder having an upper region, a lower region, and a fluid pumping chamber. The fluid inflow pump also includes a liner positioned along an inner surface of the cylinder, a piston rod extending within at least a portion of the fluid pumping chamber, wherein the piston rod is configured to pump fluid through a high pressure fluid supply tube. The fluid inflow pump also includes a high pressure connector having a first end region and a second end region, wherein the second end is coupled to the lower region of the cylinder and a high pressure seal assembly is positioned between the second end of the high pressure connector and the cylinder, and wherein the high pressure seal assembly includes a T-plate and a crush washer.

The above summary of some example embodiments is not intended to describe each disclosed embodiment or every implementation of the disclosure.

### BRIEF DESCRIPTION OF THE DRAWINGS

The disclosure may be more completely understood in consideration of the following detailed description of various embodiments in connection with the accompanying drawings, in which:

FIG. 1 is a perspective view of an illustrative thrombectomy system;

FIG. 2 illustrates an example high pressure pump of a thrombectomy system;

FIG. 3 is a cross-sectional view taken along line 3-3 of FIG. 2;

FIG. 4 is an exploded view of a portion of the high pressure pump of FIG. 2;

FIG. 5 is an exploded cross-sectional view of a portion of the high pressure pump of FIG. 2;

FIG. 6A is a perspective cross-sectional view of a portion of the high-pressure pump of FIG. 2;

FIG. 6B is a perspective cross-sectional view of a portion of another embodiment of the high-pressure pump of FIG. 2;

FIG. 7 is an exploded view of a portion of the high pressure pump of FIG. 2;

FIG. 8 illustrates an exploded view of a portion of a high pressure pump; and

FIG. 9 illustrates an exploded cross-sectional view of a portion of a high pressure pump.

While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

All numeric values are herein assumed to be modified by the term “about”, whether or not explicitly indicated. The term “about” generally refers to a range of numbers that one of skill in the art would consider equivalent to the recited value (e.g., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

Thrombectomy catheters and systems may be used to remove thrombus, plaques, lesions, clots, etc. from veins or arteries. Some thrombectomy catheters may utilize high velocity saline jets in a series to entrain fluid or clot material into and through the shaft of the catheter. Other thrombectomy systems may utilize one or more pressurized saline jets which travel backwards to create a low-pressure zone and a vacuum effect, whereby the vacuum pulls clot material into and through the shaft of the catheter. Cyclically pressurizing saline to generate high velocity jets to entrain fluid into the shaft of the catheter may be generated via a high pressure pump which may be incorporated into the thrombectomy system. Accordingly, it may be desirable to design a thrombectomy system which includes a high pressure pump capable of generating high velocity saline jets while also withstanding leaks. Thrombectomy systems which include a high pressure pump capable of generating high velocity saline jets while also withstanding leaks are disclosed herein.

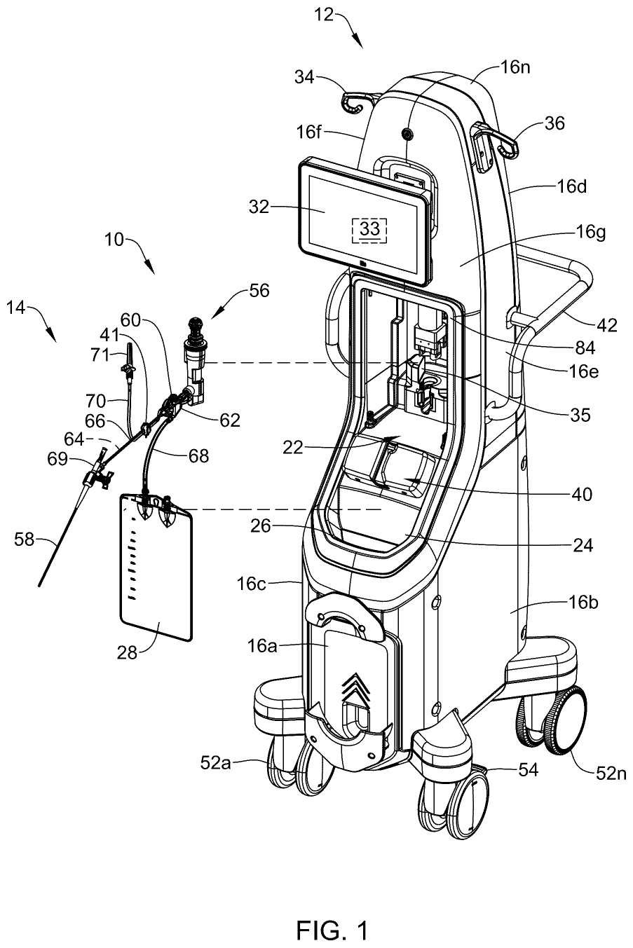

FIG. 1 is a perspective view of an illustrative thrombectomy system 10. The thrombectomy system 10 may include a control console 12, including a drive unit, and a pump/catheter assembly 14. In some instances, the pump/catheter assembly 14 may be a disposable single use device in which a new pump/catheter assembly 14 may be used with the console 12 for each medical procedure. The console 12 may include a housing enclosing the internal structure of the console 12. Shown on the console 12 are a plurality of removable panels 16a-16h about and along the console 12 enclosing the internal structure of the console 12. An illustrative console 12 is described in commonly assigned U.S. Pat. No. 7,935,077, titled THROMBECTOMY CATHETER DEPLOYMENT SYSTEM, the disclosure of which is hereby incorporated by reference. Centrally located in the console 12 and aligned to the lower region of the panel 16g may be automatically opening loading bay door assemblies (not explicitly shown) which open to expose the interior of the console 12 to provide access to a carriage assembly 22 and close during use of the pump/catheter assembly 14. Illustrative loading bay doors and assemblies are described in commonly assigned U.S. Patent Application No. 63/452,517, titled THROMBECTOMY SYSTEM WITH LOADING BAY DOORS, the disclosure of which is hereby incorporated by reference.

The console 12 may include a catch basin or drip tray 24 for collecting fluid leakage from the components of the pump/catheter assembly 14. In some instances, the drip tray 24 may be removable. Other configurations of catch basins are also contemplated. The drip tray 24 and/or a receptacle 26 may collectively support and accommodate an effluent collection bag, such as effluent collection bag 28 of the pump/catheter assembly 14. In other instances, the console 12 may include a different structure, such as a hook for hanging the effluent collection bag 28 from, or a shelf for setting the effluent collection bag 28 on. The effluent waste tube 68 may also be positioned in the roller pump 40 between the tube guides with the effluent collection bag 28 connected to the effluent waste tube 68. The effluent collection bag 28 may be suitably positioned for collecting effluent during the medical procedure. Pump rollers (not shown) of the roller pump 40 may rotatably engage the effluent waste tube 68 to control effluent fluid flow through the effluent waste tube 68 to the effluent collection bag 28.

In instances where the carriage assembly 22 is movable, a carriage assembly activation switch (not explicitly shown) may be provided with the console 12, such as located on a panel 16g, to selectively position the carriage assembly 22 inwardly or outwardly. In other instances, the carriage assembly 22 may be positioned or moved using a control panel and/or user interface 32. A user interface 32, including memory and/or processing capabilities, may be provided with the console 12, such as located at the upper region of the console 12 between the upper regions of the upper side panels 16e and 16f. The user interface 32 may be a guided user interface (GUI) including a touch screen display to allow a user to provide input to the user interface 32 and view information on a same display screen. However, this is not required. In other instances, the user input may be separate from the display screen.

Saline bag hooks 34 and 36 may extend through the panels 16e and 16f to hang saline bags therefrom. The console 12 may include a handle 42 as well as a plurality of wheels 52a-52b and brake pedals 54 for wheel lockage to assist in maneuvering the console 12 by medical personnel.

In FIG. 1, the pump/catheter assembly 14 is shown detached from the console 12. The pump/catheter assembly 14 includes an inflow pump 56 and a thrombectomy device 58. In some embodiments, the inflow pump 56 may be configured to provide fluid inflow through the thrombectomy device 58. During use, a portion of the pump/catheter assembly 14 may be secured within a portion of the console 12. In some embodiments, the pump/catheter assembly 14 may include a bubble trap 60 attached to the inflow pump 56, a connection manifold assembly 62 connected to the bubble trap 60, a fixture 41, an effluent return tube 66 connected between the connection manifold assembly 62 and the thrombectomy device 58, a high-pressure fluid supply tube 64 attached between the output of the inflow pump 56 and the thrombectomy device 58 which may be coaxially arranged inside the effluent return tube 66, a transition fixture 69 between the distal end of the effluent return tube 66 and the proximal end of the thrombectomy device 58, an effluent waste tube 68 connecting the effluent collection bag 28 to the connection manifold assembly 62, and a fluid supply tube 70 having a bag spike 71 connecting a fluid supply bag (e.g., a saline bag) (not explicitly shown) to the connection manifold assembly 62. The fluid supply tube 70 may be in fluid communication with the interior of the bubble trap 60 to provide fluid from the fluid supply bag to the inflow pump 56 and then to the thrombectomy device 58 through the high-pressure fluid supply tube 64.

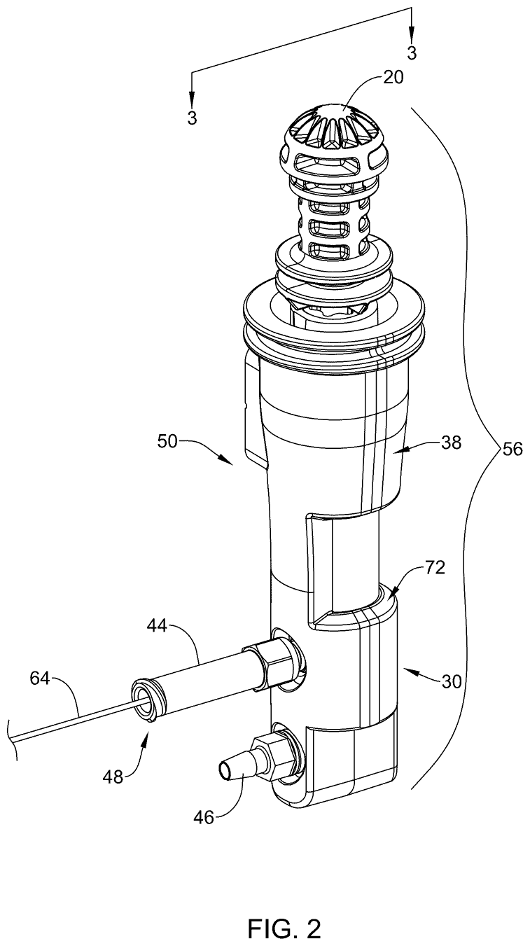

The console 12 may include a reciprocating linear actuator 84 configured to engage a pump piston head 20 (see, for example, FIG. 2) of the inflow pump 56 when the inflow pump 56 is engaged with the carriage assembly 22. The reciprocating linear actuator 84 may be disposed within the interior of the drive unit 12 and may be aligned with the pump piston head 20 (e.g., FIG. 2) when the inflow pump 56 is disposed within the interior of the drive unit 12. The reciprocating linear actuator 84 may be actuated such that reciprocating (e.g., up and down) strokes of the reciprocating linear actuator 84 drive the inflow pump 56 in response to activation of a user activation switch (not explicitly shown) or a control command from a controller 33. In some embodiments, the user activation switch may be a foot switch. In some embodiments, when the user activation switch is depressed, the reciprocating linear actuator 84 and/or the inflow pump 56 is activated and/or runs, and when the user activation switch is released, the reciprocating linear actuator 84 and/or the inflow pump 56 is stopped and/or ceases operation.

The console 12 may include a controller 33 in electronic communication with the user interface 32, the reciprocating linear actuator 84, the inflow pump 56, and/or the thrombectomy device 58. In some cases, the controller 33 may be a part of or otherwise incorporated into the user interface 32. In some embodiments, the console 12 and/or the controller 33 may include and/or be in communication with a data acquisition device 35. In some embodiments, the data acquisition device 35 may be disposed within the interior of the drive unit 12. For example, the data acquisition device 35 may be positioned on or near the carriage assembly 22. In some embodiments, the data acquisition device 35 may be configured for wireless communication. Other configurations are also contemplated. The data acquisition device 35 may be a radiofrequency identification reader and/or a barcode reader. Other types of data acquisition devices 35 may be used, as desired. In some embodiments, the user activation switch may be in electronic communication with the drive unit 12 and/or the controller 33. In some embodiments, the user activation switch may be in electronic communication with the drive unit 12 and/or the controller 33 via a wire or cable. In some embodiments, the user activation switch may be in electronic communication with the drive unit 12 and/or the controller 33 wirelessly.

FIG. 2 is a perspective view of the pump/catheter assembly 14 (e.g., FIG. 1) generally including the inflow pump 56, a high pressure connection member 44, and a low pressure inlet port 46. The inflow pump 56 may include a pump body 50. The pump body may include an upper portion 38 and a base 30. Further, the pump body 50 may include one or more features designed to engage with the carriage assembly 22. For example, the pump body 50 may include an annular surface 72 positioned along an upper portion of the base 30, whereby the annular surface is designed to engage with the carriage assembly 22 (e.g., FIG. 1) to retain the inflow pump 56 within and/or in engagement with the carriage assembly 22. The base 30 and the upper portion 38 may be molded or otherwise suitably constructed to engage with the carriage assembly 22 (e.g., FIG. 1) to retain the inflow pump 56 within and/or in engagement with the carriage assembly 22, for example.

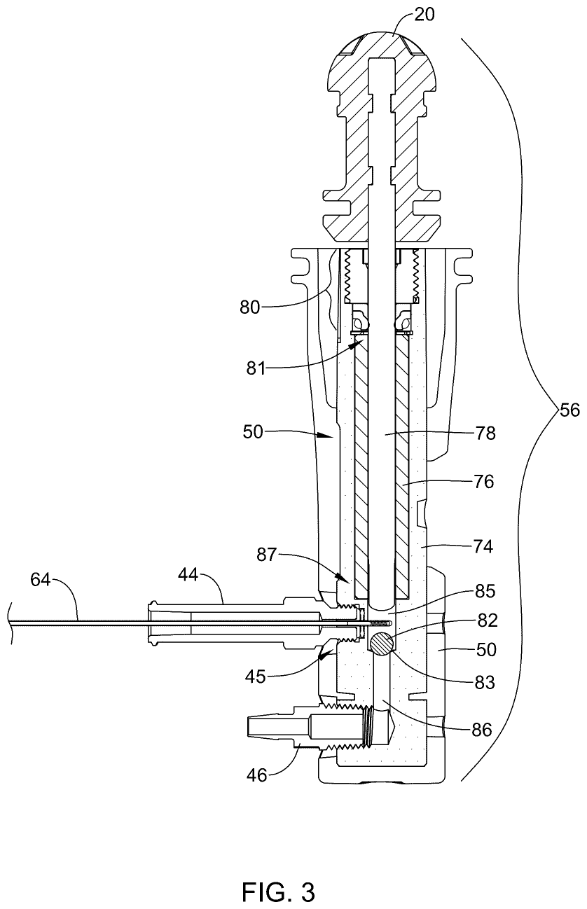

Additionally, the high pressure connection member 44 may include a first threaded connection 45 (shown in FIG. 3) attached to the pump body 50 and a second threaded connection 48 (e.g., a Luer style connection) opposite the first threaded connection 45. FIG. 2 illustrates the second threaded connection 48 including a Leur style connection, however, this is not intended to be limiting. Rather, it is contemplated that the high pressure connection member 44 may include any suitable connection which would permit the high pressure connection member 44 to be coupled to one or more pump/catheter assembly components. For example, it can be appreciated from the discussion herein that the threaded connection 48 may be designed to the connection manifold assembly 62 described herein. Further, the low pressure inlet port 46 may be designed to couple to one or more low pressure fluid supply components (e.g., fluid supply tubing) which, together with the low pressure inlet port 46 may permit a low pressure fluid (e.g., saline) to flow from a fluid supply bag to the inflow pump 56 and then to the thrombectomy device 58 through the high-pressure fluid supply tube 64.

In some embodiments, a data plate may also be included on the pump/catheter assembly 14, such as on the upper portion 38 of the pump body 50 for example, for the inclusion of a barcode, a radiofrequency identification (RFID) tag, a data storage chip, informational displays, etc. to store, communicate, and/or otherwise determine specifications and/or operational parameters associated with the pump/catheter assembly 14, the thrombectomy device 58, the inflow pump 56, etc. and/or components thereof. In at least some embodiments, the data acquisition device 35 (e.g., FIG. 1) may be configured to communicate with the data plate (e.g., the barcode, the RFID tag, the data storage chip, etc.), the pump/catheter assembly 14, the thrombectomy device 58, the inflow pump 56, etc. to obtain specifications and/or operational parameters associated with the pump/catheter assembly 14, the thrombectomy device 58, the inflow pump 56, etc., and/or components thereof. In some embodiments, the data acquisition device 35 (e.g., FIG. 1) 30 may be configured to communicate with the data plate (e.g., the barcode, the RFID tag, the data storage chip, etc.), the pump/catheter assembly 14, the thrombectomy device 58, the inflow pump 56, etc. to obtain identifying information related thereto which is associated with specifications and/or operational parameters stored in the memory of the user interface 32 and/or the controller 33. The identifying information may be used by the user interface 32 and/or the controller 33 to access the specifications and/or operational parameters associated with the pump/catheter assembly 14, the thrombectomy device 58, the inflow pump 56, etc. in use that is stored in the memory.

FIG. 3 is a cross-sectional view taken along line 3-3 of FIG. 2. As discussed above, FIG. 3 illustrates the inflow pump 56 and both the high pressure connection member 44 and the low pressure inlet port 46 attached to the pump body 50.

The pump body 50 may be an insert molded body formed of a polymeric material such as glass filled nylon (e.g., 14% glass filled nylon) or other suitable materials, to provide structural integrity for the pump 56. A non-limiting list of polymeric materials that may be used to form the pump body is disclosed herein. FIG. 3 further illustrates that the pump 56 may include an inner cylinder 74 positioned radially inward of the pump body 50. It can be appreciated that the pump body 50 may be over-molded onto the inner cylinder 74. In some examples, the inner cylinder 74 may be formed from a polymeric material. Example polymers that may be used to construct the inner cylinder 74 may include, but are not limited to an Ultra High Molecular Weight Polyethylene (UHMWPE) or other suitable materials, to provide structural integrity for the inner cylinder 74. A non-limiting list of polymers that may be used to form the inner cylinder 74 is disclosed herein. It can be appreciated that forming the inner cylinder 74 from a polymer may permit the structural features of the inner cylinder 74 to withstand deformation due to cyclical stress imparted during the operation of the pump 56.

FIG. 3 illustrates that the inner cylinder 74 may include a first threaded inlet designed to engage the first threaded connection 45 of the high pressure connection member 44 described herein. Additionally, FIG. 3 illustrates that the inner cylinder 74 may also include a second threaded inlet designed to engage a threaded portion of the low pressure inlet port 46 described herein.

FIG. 3 further illustrates that the inner cylinder 74 may further include check ball seat 83 located in a lower region of the cylinder 74 which accommodates an inlet check ball 82. The check ball seat 83 may be precisely machined to best accommodate the inlet check ball 83 for proper sealing during the pressurization stroke of a piston rod 78. The mutual contacting of the check ball 82 and the precision machining of the surface of the check ball seat 83 ensures a reliable seal of the check ball 82 to the check ball seat 83 during the downstroke of the piston rod 78.

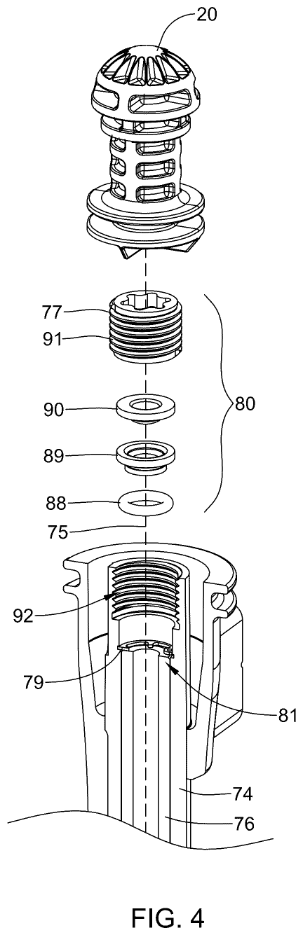

FIG. 3 further illustrates that the check ball seat 83 may form a portion of the lower region of fluid pumping chamber 85 (e.g., fluid lumen, piston chamber, inner pumping chamber, inner fluid cavity) of the inner cylinder 74. It can be further appreciated that, in some examples, the inner cylinder 74 may include a liner 76 extending along a portion of the inner cylinder 74. As shown in FIG. 3, the liner 76 may be attached to an inner surface of the inner cylinder 74. Further, the liner 76 may include a first end 81 positioned adjacent to a piston seal assembly 80 (shown in FIGS. 4-6), a second end 87 positioned adjacent to the high pressure connection member 44 and a medial portion extending between the first end 81 and the second end 87. It can be appreciated that the liner 76 may include an inner cavity designed to permit the piston rod 78 to actuate therein. Due to the potential vigorous action of the piston rod 78 over time, it can be further appreciated that the liner 76 may need to be designed from a material which provides sufficient surface friction to permit the piston rod 78 to easily actuate (e.g., slide) within the liner 76 while also providing a sufficient radial compressive seal onto the piston rod 78 over time. Further, the robustness of the material used to construct the liner 76 may permit the liner 76 to withstand the cyclical stress imparted onto the piston rod 78 over time, thereby allowing the liner 76 to sufficiently seal against the piston rod 78 and prevent leakage during operation of the pump 56 over time. The material used to construct the liner 76 may include polymer materials such as Ultra-High Molecular Weight Polyethylene (UHMWPE), acetal, PEEK or other suitable materials to provide structural integrity for the liner 76.

FIG. 3 further illustrates that a passage 86 may extend from the check ball seat 83 and through the low pressure inlet port 46. The piston rod 78 may actuate within the interior of the liner 76 to provide for intake of saline through the low pressure inlet port 46 during upstroke movement and for pressurization of saline through the high pressure supply line 64 during downstroke movement in concert with the seating of the inlet check ball 82 into the check ball seat 83.

FIG. 3 illustrates that the pump 56 may further include a piston seal assembly 80 positioned adjacent to the first end 81 of the liner 76. As will be discussed in greater detail herein, the piston seal assembly 80 may nest within an upper region of the inner cylinder 74 whereby the piston seal assembly 80 is designed to seal against the piston rod 78. It can be appreciated that the piston seal assembly 80 may be designed to maintain sealing contact between the piston rod 78 and inner cylinder 74. As discussed herein, movement of the piston rod 80 may generate high pressure forces on one or more components of the piston seal assembly 80. Therefore, the components of the piston seal assembly 80 may need to be designed to withstand high pressure forces of about 96.5 Mpa (14,000 psi) to about 151.7 MPa (22,000 psi), or about 110.3 MPa (16,000 psi) to about 147.9 MPa (20,000 psi), or about 124.1 (18,000 psi) while continuing to permit the piston rod 78 to slide through the openings in the one or more components of the piston seal assembly 80 during its upstroke and downstroke.

FIG. 4 illustrates an exploded view of a portion of a high pressure pump containing the piston seal assembly 80 described with respect to FIG. 3. FIG. 4 illustrates the piston seal assembly 80 may include an O-ring 88 which is located between a snap ring 79 and a mid-seal 89. It can be appreciated that the snap ring 79 may be designed to engage a groove in the inner cylinder 74, thereby maintaining the snap ring 79 in a fixed position relative to the inner cylinder 74. Further, FIG. 4 illustrates that the snap ring 79 may be positioned adjacent to the liner 76 described herein. Accordingly, the snap ring 79 may contact the first end 81 of the linear, thereby preventing the liner 76 from moving relative to the inner cylinder 74.

FIG. 4 illustrates that the piston seal assembly 80 may further include a mid-seal 89 positioned between the O-ring 88 and a back-up ring 90. Additionally, FIG. 4 illustrates the back-up ring 90 may be positioned between the mid-seal 89 and a seal cap 91. FIG. 4 further illustrates that the seal cap 91 may include a seal cap threaded region 77 designed to engage a cylinder threaded region 92. It can be appreciated that the engagement and rotation of the seal cap (via the threads 77) with the inner cylinder 74 (via threads 92) may permit the seal cap to compress the O-ring 88, the mid-seal 89 and the back-up ring 90 between the snap ring 79 and the seal cap 91, thereby providing a seal against the piston rod 78 (omitted from FIG. 4 for clarity but represented by dashed line 75) which allows the piston seal assembly 80 to withstand high pressure forces and prevent leakage while continuing to permit the piston rod 78 to slide through the openings in the O-ring 88, the mid-seal 89, the back-up ring 90 and the seal cap 91.

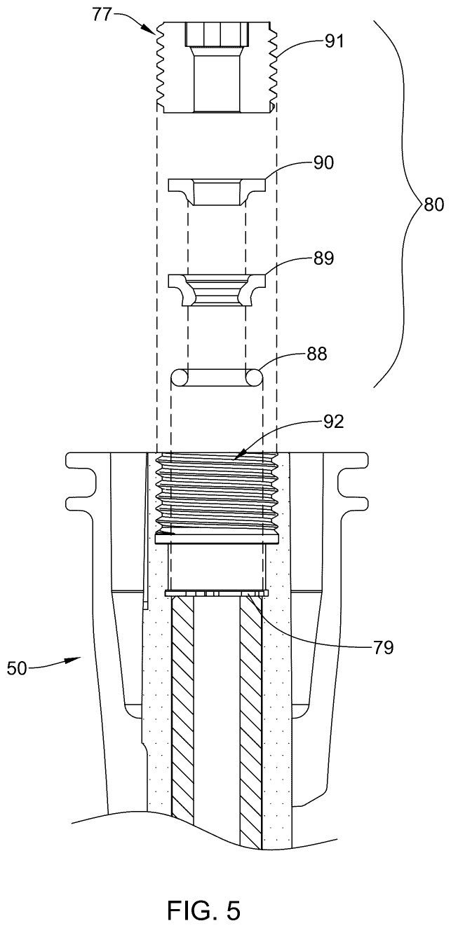

FIG. 5 illustrates an exploded cross-sectional view of a portion of the high pressure pump containing the piston seal assembly 80 vertically aligned with the upper region of the pump body 50. As discussed herein, FIG. 5 illustrates that the snap ring 79 may provide a horizontal surface upon which the O-ring 88 may rest. It can be further appreciated that a portion of the mid-seal 89 may extend into and nest within the inner aperture of the O-ring 88. Further, FIG. 5 illustrates that a portion of the back-up ring 90 may extend into and nest within the inner aperture of the mid-seal 89. Further yet, FIG. 5 illustrates that the seal cap 91 may engage an upper surface of the back-up ring 90, thereby permitting rotation of the seal cap 91 to compress the O-ring 88, the mid-seal 89 and the back-up ring 90 between the snap ring 79 and the seal cap 91.

It can be appreciated that the O-ring 88 may be formed of a material such as silicone, nitrile rubber, etc. or other suitable materials, to provide structural integrity for the O-ring 88. It can be further appreciated that the mid-seal 89 may be formed of a material such as Ultra-High Molecular Weight Polyethylene (UHMWPE), etc., or other suitable materials, configured to reduce the heat generated from an actuating piston rod 78 and provide structural integrity for the mid-seal 89. It can be further appreciated that the back-up ring 90 may be formed of a material such as polyether ether ketone (PEEK) or other suitable materials, to provide structural integrity for the back-up ring 90. It can be further appreciated that the seal cap 91 may be formed of a material such as polyether ether ketone (PEEK) or other suitable materials, to provide structural integrity for the seal cap 91.

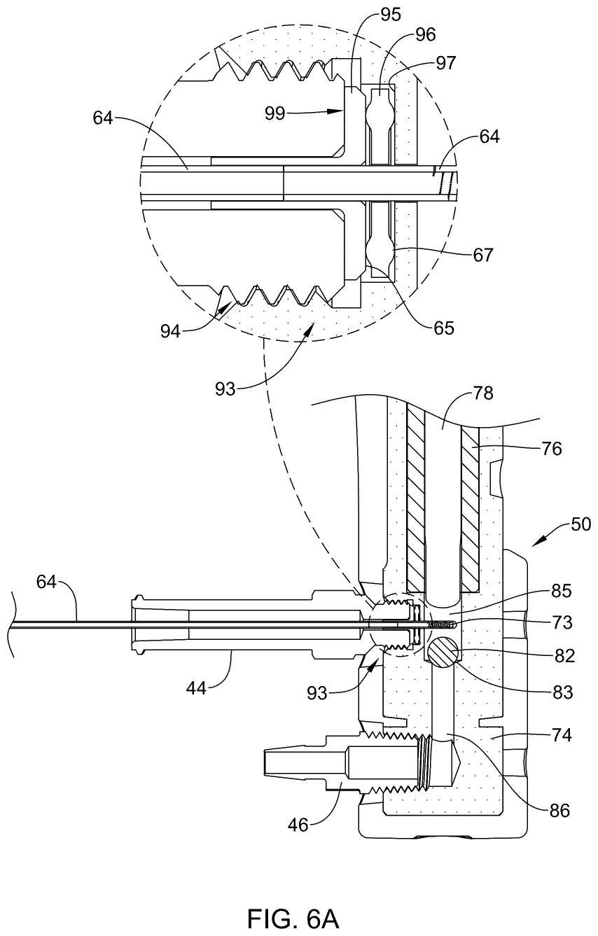

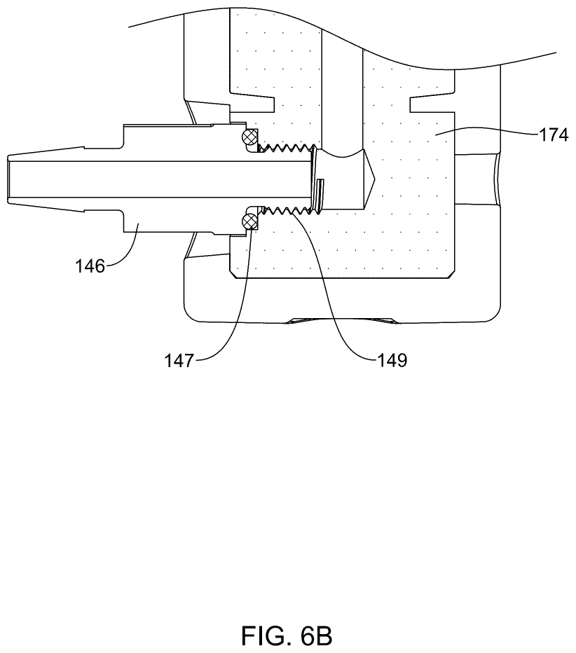

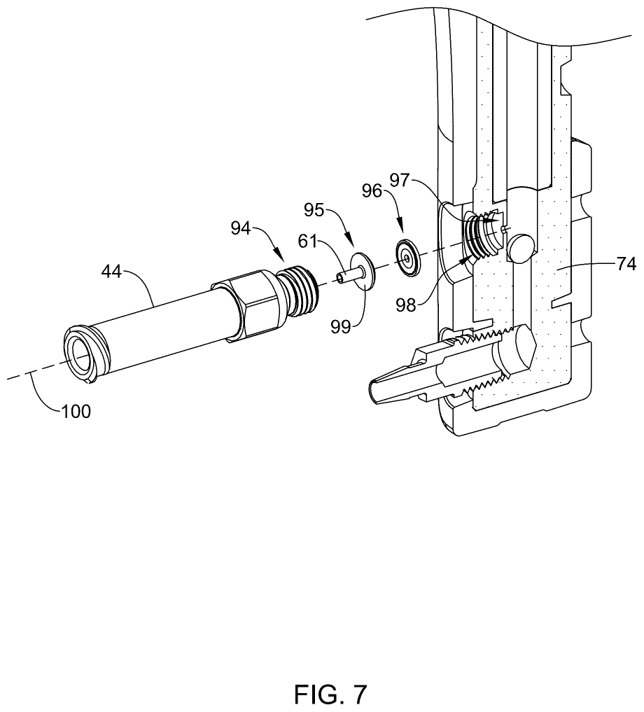

FIG. 6A illustrates a cross-sectional view of the lower region of the pump body 50, whereby the detailed view of FIG. 6A illustrates a high pressure pump containing a seal assembly 93. The high pressure seal assembly 93 may be designed to create a seal on the high pressure fluid supply line 64 which is shown extending through an inner lumen of the high pressure connection 44. As discussed herein, the high pressure connection 44 may be threaded engaged to the inner cylinder 74. For example, FIG. 7 illustrates the high pressure connection 44 may include a threaded region 94 which may be designed to engage a threaded region 98 of the inner cylinder 74.

The detailed view of FIG. 6A, together with the exploded view shown in FIG. 7, illustrate that the high pressure seal assembly 93 may include a T-plate 95 and a washer 96 (e.g., crush washer, compression washer, etc.), both of which may be designed to be positioned between the high pressure connection 44 and a lateral facing surface 97 of the inner cylinder 74. As disclosed herein, the washer 96 (e.g., crush washer) may be any annular, round, disk-shaped washer made out of a material (e.g., metal, polymer, etc.) that may be crushed, deformed, flattened, compressed, etc. to create a fluid seal around one or more structures positioned adjacent to the washer 96. Further, it can be appreciated that the washer 96 may be formed of a material such as titanium (e.g., Grade 2 titanium), stainless steel, etc. or other suitable materials, to provide structural integrity and deformability for the washer 96.

Additionally, as disclosed herein a T-plate may be a “T-shaped” member including a tubular member attached to a flange disposed at an end of the tubular member. For example, the detailed view of FIG. 6 illustrates the T-plate 95 may include a flange having a first radially outward facing surface 99 and a second radially inward facing surface 65, whereby the flange is positioned between the threaded region 94 of the high pressure connection 44 and the washer 96. The first radially outward facing surface 99 may be designed to contact an end face of the threaded region 94 of the high pressure connection 44, while the second radially inward facing surface 65 may be designed to contact the lateral facing surface 97 of the inner cylinder 74. Additionally, FIG. 7 illustrates that the T-plate 95 may include a stem 61 extending away from the first radially outward facing surfacing surface 99 of the flange. In some examples, the stem 61 may be designed to extend into at least a portion of an inner lumen of the high pressure connection 44. Further, the detailed view of FIG. 6 further illustrates that the washer 96 may be positioned between the T-plate 95 and the lateral facing surface 97 of the inner cylinder 74.

FIG. 7 illustrates that both the T-plate 95 and the washer 96 may each include a central aperture designed to permit the high pressure fluid supply line 64 to extend therethrough and into the fluid pumping chamber 85 of the inner cylinder 74. Further, the detailed view of FIG. 6 illustrates that the washer 96 may include deformable portion 67 designed to flatten and seal against the high pressure fluid supply line 64 as the second radially inward facing surface 65 of the T-plate 95 is pushed against the deformable portion 67 of the washer 96 as the high pressure connection 44 is screwed into the inner cylinder 74.

FIG. 6A and FIG. 7 illustrate that the high pressure fluid supply line 64 may include a filter 73 positioned on the end of the high pressure fluid supply line 64 which extends into the fluid pumping chamber 85 of the inner cylinder 74. It can be appreciated that as the piston rod 78 moves through an upstroke it may draw fluid (e.g., saline) through the low pressure inlet port 46, through the fluid pathway 86 whereby the fluid pushes the check ball 82 upward, allowing the fluid to enter the fluid pumping chamber 85. Further, as the piston rod 78 moves through a downstroke, the fluid which had been drawn into the fluid pumping chamber 85 may push the check ball 82 into the check ball seat 83 (designed to receive the check ball), thereby sealing and preventing the fluid from entering the fluid pathway 86. Further, the pressure exerted on the fluid via the downstroke of the piston 78 may force the fluid into the filter 73 and into the high pressure fluid supply line 64 whereby the fluid travels through the high pressure fluid supply line 64 and into the thrombectomy catheter 58. The high pressure seal assembly 93 (including the T-plate 95 and washer 96) may prevent leakage through the inner cylinder 74 and/or pump body 50 while maintaining high pressure as the piston rod 78 continues to cycle and push fluid through the high pressure fluid supply line 64. In some examples, the filter 73 may include a plurality of filter openings (e.g., holes, apertures, orifices, etc.) extending from an inner surface of the high pressure fluid supply line 64 to an outer surface of the high pressure fluid supply line 64 (e.g., through the wall of the high pressure fluid supply line 64). In some examples, the diameter of the filter openings of the filter 73 may be about 0.0102 millimeters (mm) (0.0004 inches) to about 0.0457 mm (0.0018 inches), or about 0.0152 mm (0.0006 inches) to about 0.0406 mm (0.0016 inches), or about 0.0203 mm (0.0008 inches) to about 0.0356 mm (0.0014 inches), or about 0.0254 mm (0.0010 inches) to about 0.0305 mm (0.0012 inches), or about 0.0305 mm (0.0012 inches), or about 0.0254 mm (0.0010 inches).

FIG. 6B illustrates another example cross-sectional view of the lower region of the pump body 50, whereby FIG. 6B illustrates another example low pressure inlet port 146 (the low pressure inlet portion 146 may be similar in form and function to the low pressure inlet port 46 described herein). As discussed above, the low pressure inlet port 146 may be designed to couple to one or more low pressure fluid supply components (e.g., fluid supply tubing) which, together with the low pressure inlet port 146 may permit a low pressure fluid (e.g., saline) to flow from a fluid supply bag to the inflow pump 56 (shown in FIG. 1) and then to the thrombectomy device 58 (shown in FIG. 1) through the high-pressure fluid supply tube 64 (shown in FIG. 1). As illustrated in FIG. 6B, the low pressure inlet portion 146 may include a threaded region 149 which is configured to engage a threaded region of an inner cylinder 174 (the inner cylinder 174 may be similar in form and function the inner cylinder 74 illustrated in FIG. 3). Additionally, FIG. 6B illustrates that an O-ring 147 may be positioned between a portion of the low pressure inlet port 146 and the inner cylinder 174, whereby compression of the O-Ring 147 is configured to provide a reliable seal between the low pressure inlet portion 146 and the inner cylinder 174.

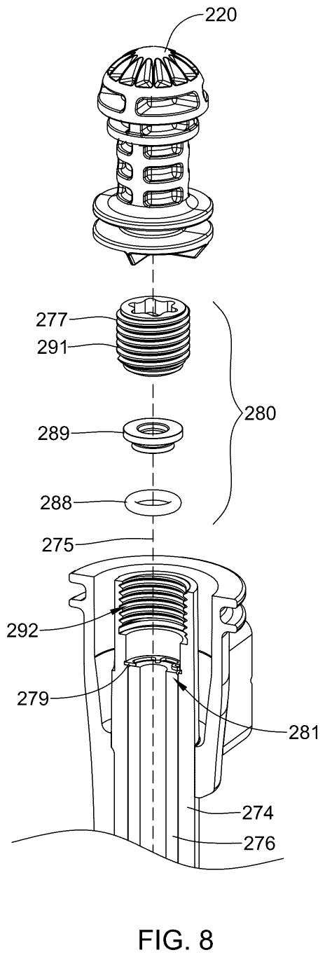

FIG. 8 illustrates an exploded view of a portion of a high pressure pump containing a piston seal assembly 280. The piston seal assembly 280 may be similar in form and function to the piston seal assembly 80 described herein. For example, FIG. 8 illustrates the piston seal assembly 280 may include an O-ring 288 which is located between a snap ring 279 and a mid-seal 289. It can be appreciated that the snap ring 279 may be designed to engage a groove in the inner cylinder 274, thereby maintaining the snap ring 279 in a fixed position relative to the inner cylinder 274. Further, FIG. 8 illustrates that the snap ring 279 may be positioned adjacent to a liner 276 (similar in form and function to the liner 76 described herein). Accordingly, the snap ring 279 may contact the first end 281 of the liner, thereby preventing the liner 276 from moving relative to the inner cylinder 274 (similar in form and function to the liner 74 described herein).

FIG. 8 illustrates that the piston seal assembly 280 may further include a mid-seal 289 positioned between the O-ring 288 and a seal cap 291. FIG. 8 further illustrates that the seal cap 291 may include a seal cap threaded region 277 designed to engage a cylinder threaded region 292. It can be appreciated that the engagement and rotation of the seal cap (via the threads 277) with the inner cylinder 274 (via threads 292) may permit the seal cap to compress the O-ring 288 and the mid-seal 289 between the snap ring 279 and the seal cap 291, thereby providing a seal against the piston rod 78 (shown in FIG. 3 but omitted from FIG. 8 for clarity but represented by dashed line 275) which allows the piston seal assembly 280 to withstand high pressure forces and prevent leakage while continuing to permit the piston rod 78 to slide through the openings in the O-ring 288, the mid-seal 289, and the seal cap 291.

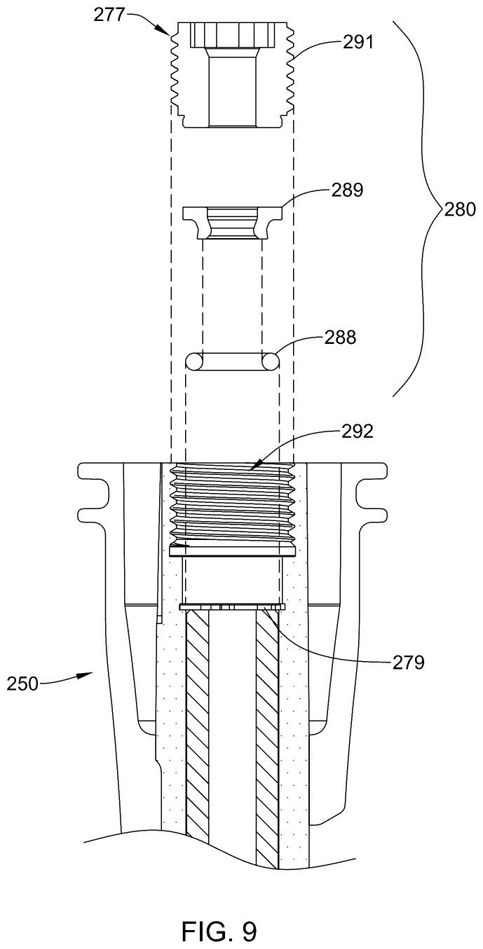

FIG. 9 illustrates an exploded cross-sectional view of a portion of a high pressure pump containing a piston seal assembly 280 vertically aligned with the upper region of a pump body 250. As discussed herein, FIG. 9 illustrates that the snap ring 279 may provide a horizontal surface upon which the O-ring 288 may rest. It can be further appreciated that a portion of the mid-seal 289 may extend into and nest within the inner aperture of the O-ring 288. Further yet, FIG. 9 illustrates that the seal cap 291 may engage an upper surface of the mid-seal 289, thereby permitting rotation of the seal cap 291 to compress the O-ring 288 and the mid-seal 289 between the snap ring 279 and the seal cap 291.

It can be appreciated that the O-ring 288 may be formed of a material such as silicone, nitrile rubber, etc. or other suitable materials, to provide structural integrity for the O-ring 288. It can be further appreciated that the mid-seal 289 may be formed of a material such as Ultra-High Molecular Weight Polyethylene (UHMWPE), etc., or other suitable materials, configured to reduce the heat generated from an actuating piston rod 78 (shown in FIG. 3 but omitted from FIG. 9 for clarity) and provide structural integrity for the mid-seal 289. It can be further appreciated that the seal cap 291 may be formed of a material such as polyether ether ketone (PEEK) or other suitable materials, to provide structural integrity for the seal cap 291.

The materials that can be used for the various components of the thrombectomy catheter, pump/catheter assembly, and/or other devices disclosed herein may include those commonly associated with medical devices. For simplicity purposes, the following discussion refers to the pump/catheter assembly and its related components. However, this is not intended to limit the devices and methods described herein, as the discussion may be applied to other similar devices, tubular members and/or components of tubular members or devices disclosed herein.

The various components of the thrombectomy catheter, pump/catheter assembly, and/or other devices disclosed herein may include a metal, metal alloy, polymer (some examples of which are disclosed herein), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

The various components of the thrombectomy catheter, pump/catheter assembly, and/or other devices disclosed herein may include a polymer. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In at least some embodiments, portions or all of the thrombectomy catheter, thrombectomy catheter, pump/catheter assembly, and/or other devices disclosed herein pump/catheter assembly, and/or other devices disclosed herein may be doped with, made of, or otherwise include a radiopaque material. Radiopaque materials are understood to be materials capable of producing a relatively bright image on a fluoroscopy screen or another imaging technique during a medical procedure. This relatively bright image aids the user of the pump/catheter assembly and its related components in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the pump/catheter assembly and its related components to achieve the same result.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A high pressure pump for use in thrombectomy system, comprising:
a cylinder having an upper region, a lower region, and a fluid pumping chamber;
a piston rod extending within at least a portion of the fluid pumping chamber, wherein the piston rod is configured to pump fluid through a high pressure fluid supply tube;
a high pressure connector having a first end region and a second end region, wherein the second end is coupled to the lower region of the cylinder; and
a high pressure seal assembly positioned between the second end of the high pressure connector and the cylinder, and wherein the high pressure seal assembly includes a T-plate and a crush washer.

2. The high pressure pump of claim 1, wherein the T-plate is positioned radially outward of the crush washer.

3. The high pressure pump of claim 2, wherein the T-plate includes a first radially outward facing surface and a second radially inward facing surface, and wherein the crush washer is positioned between the second radially inward facing surface and the cylinder.

4. The high pressure pump of claim 3, wherein the first radially outward facing surface of the T-plate is configured to engage the high pressure connector.

5. The high pressure pump of claim 4, wherein the second end region of the high pressure connector includes a first threaded region, and wherein the first threaded region of the high pressure connector is configured to engage a second threaded region of the cylinder, and wherein rotation of the first threaded region of the high pressure connector along the second threaded region of the cylinder engages the second radially inward facing surface of the T-plate with the crush washer.

6. The high pressure pump of claim 5, wherein the engagement of the second radially inward facing surface of the T-plate with the crush washer deforms the crush washer from a first configuration to a second configuration different from the first configuration.

7. The high pressure pump of claim 6, wherein the crush washer includes a first central aperture, and wherein the high pressure fluid supply tube extends through the first central aperture of the crush washer, and wherein the deformation of the crush washer from the first configuration to the second configuration seals the crush washer onto the high pressure fluid supply tube.

8. The high pressure pump of claim 7, wherein a proximal end region of the high pressure fluid supply includes a filter and wherein at least a portion of the filter is positioned within the fluid pumping chamber.

9. The high pressure pump of claim 8, wherein the cylinder further includes a check ball seat configured to receive a check ball positioned within the fluid pumping chamber, and wherein the filter is positioned between the check ball and the piston rod.

10. The high pressure pump of claim 1, wherein the crush washer includes a first cross-sectional thickness and a second cross-sectional thickness different from the first cross-sectional thickness.

11. The high pressure pump of claim 10, wherein the T-plate includes a stem having a lumen, and wherein the high pressure fluid supply tube is configured to extend within the lumen of the stem.

12. The high pressure pump of claim 11, wherein at least a portion of the stem is configured to extend into a lumen of the high pressure connector.

13. The high pressure pump of claim 1, further comprising a liner extending along at least a portion of an inner surface of the cylinder.

14. The high pressure pump of claim 13, wherein the liner is formed from ultra-high-molecular-weight polyethylene.

15. A thrombectomy system, the system comprising:
a fluid inflow pump coupled to a thrombectomy catheter and driven by a console, the fluid inflow pump comprising:
a cylinder having an upper region, a lower region, and a fluid pumping chamber;
a piston rod extending within at least a portion of the fluid pumping chamber, wherein the piston rod is configured to pump fluid through a high pressure fluid supply tube;
a high pressure connector having a first end region and a second end region, wherein the second end is coupled to the lower region of the cylinder; and
a high pressure seal assembly positioned between the second end of the high pressure connector and the cylinder, and wherein the high pressure seal assembly includes a T-plate and a crush washer.

16. The high pressure pump of claim 15, wherein the T-plate includes a first radially outward facing surface and a second radially inward facing surface, and wherein the crush washer is positioned between the second radially inward facing surface and the cylinder.

17. The high pressure pump of claim 16, wherein the second end region of the high pressure connector includes a first threaded region, and wherein the first threaded region of the high pressure connector is configured to engage a second threaded region of the cylinder, and wherein rotation of the first threaded region of the high pressure connector along the second threaded region of the cylinder engages the second radially inward facing surface of the T-plate with the crush washer.

18. The high pressure pump of claim 17, wherein the engagement of the second radially inward facing surface of the T-plate with the crush washer deforms the crush washer from a first configuration to a second configuration different from the first configuration.

19. The high pressure pump of claim 18, wherein the crush washer includes a first central aperture, and wherein the high pressure fluid supply tube extends through the first central aperture of the crush washer, and wherein the deformation of the crush washer from the first configuration to the second configuration seals the crush washer onto the high pressure fluid supply tube.

20. A thrombectomy system, the system comprising:
a fluid inflow pump coupled to a thrombectomy catheter and driven by a console, the fluid inflow pump comprising:
a cylinder having an upper region, a lower region, and a fluid pumping chamber;
a liner positioned along an inner surface of the cylinder;
a piston rod extending within at least a portion of the fluid pumping chamber, wherein the piston rod is configured to pump fluid through a high pressure fluid supply tube;
a high pressure connector having a first end region and a second end region, wherein the second end is coupled to the lower region of the cylinder; and
a high pressure seal assembly positioned between the second end of the high pressure connector and the cylinder, and wherein the high pressure seal assembly includes a T-plate and a crush washer.
